(19) United States
(12) Patent Application Publication
(10) Pub. No.: US 2015/0018299 A1
(43) Pub. Date: Jan. 15, 2015

(54) TREATMENT OF LIVER CONDITIONS

(71) Applicant: CAN-FITE BIOPHARMA LTD., Petach Tikva (IL)

(72) Inventors: Pnina Fishman, Herzliya (IL); Shira Cohen, Herzliya (IL); Sara Bar-Yehuda, Rishon Le Zion (IL)

(21) Appl. No.: 14/374,067

(22) PCT Filed: Jan. 23, 2013
(86) PCT No.: PCT/IL2013/050064

§ 371 Date: Jul. 23, 2014

Related U.S. Application Data
(60) Provisional application No. 61/589,430, filed on Jan. 23, 2012.
(60) Provisional application No. 61/752,118, filed on Jan. 14, 2013.

(51) Int. Cl. A61K 31/7076

(52) U.S. Cl. CPC A61K 31/7076
USPC 514/46

(57) ABSTRACT

The present disclosure concerns the A₃ adenosine receptor agonist, 2-Chloro-N⁶-(3-iodobenzyl)-adenosine-5′-N-methyluronamide (Cl-IB-MECA) in an amount of at least about 10 mg/day, for use in treatment of hepatocellular carcinoma (HCC). The present invention also provides Cl-IB-MECA for use in maintenance of liver function in a subject having a chronic liver disease, such as cirrhosis. The liver function is considered as maintained if level of at least one physiological parameter indicative of liver function is essentially constant between two or more time points, i.e. the difference between the two time points does not exceed a medically acceptable tolerance.

### TECHNOLOGICAL FIELD

This invention relates to maintenance of liver function in subjects having chronic liver conditions and to the treatment of liver cancer.

### PRIOR PUBLICATIONS

The following is a list of prior publications considered to be relevant as background to the current disclosure:
Madi L, et al. (2007). Over-expression of A3 adenosine receptor in PBMNC of rheumatoid arthritis patients: involvement of NF-κB in mediating receptor level. J Rheumatolo 34:20-26
Ochaion A, et al. (2009). The anti-inflammatory target A3 adenosine receptor is over-expressed in rheumatoid arthritis, psoriasis and Crohn's disease. Cell Immunol 258:115-122
Fishman P, et al. (2006). The PI3K-NF-κB signal transduction pathway is involved in mediating the anti inflammatory effect of IB-MECA in adjuvant-induced arthritis. Arth Res Ther 8:R33
Bar-Yehuda S, et al. (2007). The anti-inflammatory effect of A3 adenosine receptor agonists: a novel targeted therapy for rheumatoid arthritis. Expert Opin Inves Drugs 16:1601-1613
WO 2009/050707
WO 2007/043054
Gregorio Gomez and Michall V Sitkovsky (2003). Differential requirement for A2a and A3 adenosine receptors for the protection effect of inosine in vivo Blood 102(13):4472-4478
Bar-Yehuda S., et al. (2008). The A3 adenosine receptor agonist CF102 induces apoptosis of hepatocellular carcinoma via de-regulation of the Wnt and NF-kB signal transduction pathways. Int J Onc 33: 287-295, 2008
H. Thomas Lee, et al, (2006). A₃ adenosine receptor activation decreases mortality and renal and hepatic injury in murine septic peritonitis Am J Physiol Regul Integr Comp Physiol 291: R959-R969

### BACKGROUND

The Gi protein associated cell surface A₃ adenosine receptor (A₃AR), is over-expressed in inflammatory cells and in peripheral blood mononuclear cells (PBMCs) derived from patients with various auto-immune inflammatory diseases, such as rheumatoid arthritis psoriasis and Crohn's Disease [Madi L, et al. (2007), Over-expression of A3 adenosine receptor in PBMNC of rheumatoid arthritis patients: involvement of NF-κB in mediating receptor level. J Rheumatolo 34:20-26; Ochaion A, et al. (2009), The anti-inflammatory target A3 adenosine receptor is over-expressed in rheumatoid arthritis, psoriasis and Crohn's disease. Cell Immunol 258:115-122].

Activation of the receptor with highly specific agonists, such as IB-MECA and Cl-IB-MECA, induce a marked anti-inflammatory effect, via down-regulation of the NF-κB signaling pathway, resulting in de-regulation of pro-inflammatory cytokines and induction of inflammatory cells apoptosis [Fishman P, et al. (2006) The PI3K-NF-κB signal transduction pathway is involved in mediating the anti inflammatory effect of IB-MECA in adjuvant-induced arthritis. Arth Res Ther 8:R33; Bar-Yehuda S, et al. (2007) The anti-inflammatory effect of A3 adenosine receptor agonists: a novel targeted therapy for rheumatoid arthritis. Expert Opin Inves Drugs 16:1601-1613.

Methods and compositions for inducing hepatocyte proliferation and liver regeneration, making use of an A₃AR agonist, e.g. Cl-IB-MECA, is described in International Patent Application Publication No. WO 2009/050707.

In addition, methods for monitoring and determining disease state in patients having liver cancer is described in International Patent Application Publication No. WO2007/043054, the method is based on determining level of expression of A3 adenosine receptor in white blood cells, the level of expression being indicative of disease state. Gomez G. and Sitkovsky M describe the effect of inosine, an endogenous nucleoside known to specifically bind to A₃AR, in protecting against ConA-induced hepatitis [Gregorio Gomez and Michall V Sitkovsky (2003) Differential requirement for A2a and A3 adenosine receptors for the protection effect of inosine in vivo Blood 102(13):4472-4478].

In addition, the protection effect of Cl-IB-MECA in Con. A induced acute liver inflammation was described by Cohen et al, 2011. [Cohen et al. Journal of cellular physiology September 2011 pp. 2438-2447]. The effect of Cl-IB-MECA in inhibiting HCC (hepatocellular carcinoma) tumor growth in rats in a dose-dependent manner was shown in Bar Yehuda et al., 2008 [Bar-Yehuda et al., Int. J. Oncol. 33: 287-295].

Finally, Thomas Lee H. et al. show the effect of A₃AR activation reduce mortality and improve renal and hepatic function after CLP-induced sepsis [H. Thomas Lee, Mihwa Kim, Jin Deok Joo, George Gallos, Jiang-Fan Chen, and Charles W. Emala, (2006) A₃ adenosine receptor activation decreases mortality and renal and hepatic injury in murine septic peritonitis Am J Physiol Regul Integr Comp Physiol 291: R959-R969].

### SUMMARY OF DISCLOSURE

The present disclosure is based on findings in human patients having hepatocellular carcinoma (HCC). It was shown, in accordance with the current disclosure that (i) human patients treated with the A₃ Adenosine receptor agonist (A₃AR), 2-Chloro-N⁶-(3-iodobenzyl)-adenosine-5′-N-methyluronamide (Cl-IB-MECA) maintained an essentially steady level of some physiological parameters that are indicative of their liver function, and (ii) that certain doses of Cl-IB-MECA were effective in treating HCC as evidenced by the prolongation of survival of the HCC patients beyond expectation.

It was surprisingly found, in accordance with the current disclosure, that unlike expectation based on dose-response relationships in treatment of human subjects with another A₃AR, N⁶-(3-iodobenzyl)-adenosine-5′-N-methyluronamide (IB-MECA) as well as prior findings with Cl-IB-MECA in an experimental HCC animal model (Bar-Yehuda et al, 2008), where a bell-shaped relationship was observed, in the use of Cl-IB-MECA in human HCC patients, increase in the dose showed an increase in therapeutic activity up to a maximal effect dose in which absorption reaches close to saturation (namely administration of a higher dose will yield little or no absorption beyond the maximal effect dose).

Thus, provided by the current disclosure are aspects focusing on maintenance of liver function (each, a “maintenance aspect”) and also aspects focused on treatment of liver cancer (each, a “treatment aspect”)

In accordance with one maintenance aspect, the present disclosure provides Cl-IB-MECA for use in maintenance of liver function in a subject having a chronic liver disease. The chronic liver disease may be, but not limited to, liver cancer, including hepatocellular carcinoma (HCC) or a metastatic cancer with metastases in the liver.

Maintenance of liver function, by one embodiment, means that the level of at least one physiological parameter indicative of liver function is maintained over a period of time, e.g. for several weeks, several month or even for years. Maintenance of a liver function may be determined by measuring or otherwise determining (e.g. through medically acceptable qualitative assessment) of at least one physiological parameter in at least two different time points. Maintenance of a liver function may mean that the measured physiological parameter is substantially constant within medically acceptable tolerance.

The disclosure also provides, in accordance with another maintenance aspect, a method for maintaining liver function in a subject having a chronic liver disease, the method comprising administering to the subject having a chronic liver disease an amount of Cl-IB-MECA effective to maintain liver function at an essentially constant level.

The disclosure also provides, in accordance with a further maintenance aspect, use of an effective amount of Cl-IB-MECA for the preparation of a pharmaceutical preparation for use in maintaining liver function.

Also provided herein is a package comprising a pharmaceutical composition that comprises, as active ingredient Cl-IB-MECA and instructions for use of the Cl-IB-MECA in maintaining liver function, the instructions comprising administration to a subject having a chronic liver disease an amount of Cl-IB-MECA effective to maintain the liver function.

Disclosed herein, in accordance with the maintenance aspect, is also a pharmaceutical composition for maintaining liver function in a subject having a chronic liver disease, the composition comprises an effective amount of Cl-IB-MECA.

In the context of both aspects, the term “effective amount” or “an amount effective” should be understood to mean an amount that when administered in a defined therapeutic regimen, e.g. once, twice or thrice daily, is effective in achieving the intended therapeutic effect, which in the maintenance aspect of the invention is the maintenance of the liver function in a subject with chronic liver disease. The effective amount may, for example, be an amount of at least about 10 mg/day, e.g. any of the doses noted below in connection with the treatment aspect.

The disclosure also provides, in accordance with the treatment aspect, Cl-IB-MECA at a dose of at least about 10 mg/day (e.g. at least about 10 mg in a treatment regime of once daily treatment, at least about 5 mg twice daily, at least about 3.3 mg thrice daily, etc.) for use in treatment of HCC in human subjects.

A dose of at least about 10 mg/day may be a dose of at least about 15 mg/day, at least about 20 g/day, at least about 25 mg/day, at least about 30 mg/day, at least about 35 mg/day, at least about 40 mg/day, at least about 45 mg/day and about 50 mg/day or even higher. The total amount of Cl-IB-MECA given a day to a patient, irrespective of the number of administrations is referred to herein as a “daily treatment dose”.

A daily treatment dose of about 50 mg is one example of a dose for use in the treatment aspect. It may for example be a twice daily dose of about 25 mg each or a thrice daily dose of about 16 mg.

The disclosure also provides, in accordance with the treatment aspect, a method for treating HCC in human subject comprising administering to the subject a daily treatment dose of Cl-IB-MECA.

The disclosure also provides, by a further treatment aspect, use of Cl-IB-MECA, for the preparation of a pharmaceutical preparation for HCC treatment in a unit dosage form for administering of the daily treatment dose of at least 10 mg/day. Where the dosage form is intended for administering to a patient in a treatment regimen comprising n doses per day, then a unit dosage form may comprise 1/n portion of the daily treatment dose (e.g., where the intended daily treatment dose is 20 mg and the treatment regiment is twice daily then each unit dosage form will have a dose of 10 mg; or where the intended daily treatment dose is 50 mg and the treatment regiment is twice daily then each unit dosage form will have a dose of 25 mg).

The disclosure also provides, in accordance with the treatment aspect, Cl-IB-MECA for use in the treatment of HCC in a unit dosage form for administering to a subject in need of said treatment the daily treatment dose of said Cl-IB-MECA.

Further, in accordance with the treatment aspect, there is provided a unit dosage form comprising Cl-IB-MECA, for administering to a subject having HCC the daily treatment dose of Cl-IB-MECA.

Further provided, in accordance with the treatment aspect, is a package comprising a pharmaceutical composition comprising as active ingredient Cl-IB-MECA and instructions for use of the pharmaceutical composition for treatment of HCC, the instructions comprising administering to a subject in need of the treatment the daily treatment dose of Cl-IB-MECA.

### BRIEF DESCRIPTION OF THE FIGURES

In order to understand the invention and to see how it may be carried out in practice, embodiments will now be described, by way of non-limiting example only, with reference to the accompanying drawings, in which:

FIG. 1A-1B are graphs showing pharmacokinetic behavior of CF102 after single dose (n=3/group, FIG. 1A); and after repeated dose at steady state (n=3/group, FIG. 1B).

FIG. 2A-2G are graphs showing serum levels of liver enzymes Alanine transaminase ALT (FIG. 2A) and aspartate transaminase AST (FIG. 2B), albumin (FIG. 2C), bilirubin (FIG. 2D), prothrombin time (PT) (FIG. 2E), ALK phosphatase (FIG. 2F) and α-fetoprotein (FIG. 2G) in patients with hepatocellular carcinoma (HCC). The data revealed that the different parameters remained stable, suggesting that Cl-IB-MECA maintained the liver function.

### DETAILED DESCRIPTION

Chronic liver disease involves the gradual destruction of liver tissue over time and may be divided to cirrhosis of liver, and fibrosis of the liver. The former involves the slow replacement of normal functioning liver tissue with scar tissue, progressively diminishing blood flow through the liver, resulting in reduced or lack of processing of nutrients, hormones, drugs and poisons (bacteria and toxins). In addition, production protein, bile and other substances is inhibited. Scarring also impairs the liver ability to control infections.

As the damage to the liver in chronic liver diseases, and in particularly in cirrhosis is at times irreversible, it would be therapeutically beneficiary to at least maintain liver function during the manifestation of the disease.

The present disclosure concerns two aspects, maintenance of liver function in patients having HCC (the “maintenance aspect”) and treatment of HCC (the “treatment aspect”). Both aspects which are further detailed below make use of Cl-IB-MECA.

Cl-IB-MECA is a well recognized A₃ adenosine receptor agonist, having the trivial chemical name 1-[2-Chloro-6-[[(3-iodophenyl)methyl]amino]-9H-purin-9-yl]-1-deoxy-N-methyl-β-D-ribofuranuronamide and has the chemical formula C₁₈H₁₈ClIN₆O₄ and the chemical structure:

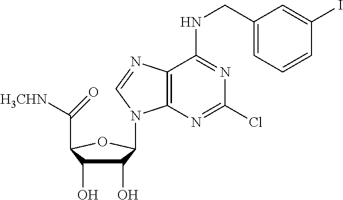

Cl-IB-MECA is known to exert its prime effect through the binding and activation of the A₃AR, namely, its affinity to the A₃AR is much higher (Ki 3 times lower) than the affinity to any other adenosine receptors.

In accordance the maintenance aspect, there is provided Cl-IB-MECA for use in maintenance of liver function in a subject having a chronic liver disease, the liver function being maintained if at least one physiological parameter indicative of liver function is essentially constant within medically acceptable tolerance, between two or more time points.

Further, within the maintenance aspect, the present disclosure provides a method for maintaining liver function in a subject having a chronic liver disease, the method comprising administering to the subject having a chronic liver disease an amount of Cl-IB-MECA effective to maintain at least one physiological parameter indicative of liver function in an essentially constant level within a medically acceptable tolerance, between at least two time points.

The disclosure also provides, in accordance with the maintenance aspect, use of an effective amount of Cl-IB-MECA for the preparation of a pharmaceutical preparation for use in maintaining liver function.

Also provided in accordance with the maintenance aspect is a package comprising a pharmaceutical composition comprising as active ingredient Cl-IB-MECA and instructions for use of the Cl-IB-MECA in maintaining liver function, the instructions comprising administration to a subject having a chronic liver disease an amount of Cl-IB-MECA effective to maintain at least one physiological parameter indicative of liver function at an essentially constant level within a medically acceptable tolerance between at least two time points.

Finally, within the maintenance aspect, there is disclosed herein a pharmaceutical composition for maintaining liver function in a subject having a chronic liver disease, the composition comprises an amount of Cl-IB-MECA being sufficient to maintain at least one physiological parameter indicative of liver function at an essentially constant level within a medically acceptable tolerance between at least two time points.

The liver function may be determined by any measurable or detectable physiological parameters indicative of same.

In some embodiment, the physiological parameter is obtained by liver imaging, such as computerized tomography (CT) scan, ultrasound, magnetic resonance imaging (MRI), liver scan, laparoscopy.

In some further embodiments, the liver physiological parameter is determined by liver biopsy.

In yet some other embodiments, the physiological parameter is one or more liver function tests (LFT). LFT are clinical biochemistry laboratory assays of blood or blood fraction (preferably serum). LFT include blood enzymes assays, such as and without being limited thereto, alanine aminotransferase (ALT), aspartate aminotransferase (AST), alkaline phosphate (ALP), gamma-glutamyl transferase (GGT), Lactate dehydrogenase (LDH); LFT also includes blood total bilirubin assay (measuring the yellow bilirubin pigment in the blood); blood albumin level, total protein (albumin and antibodies); blood glucose level.

The liver function may be determined by one or the combination of two or more of the determined physiological parameters.

In accordance with the maintenance aspect, a difference in the level of one or more physiological parameters is determined between two time points. It is an object of any treatment that the liver function is essentially maintained constant, thus, between such two or more time points it is desired that the physiological parameter is maintained essentially constant or the different is maintained within a medically acceptable tolerance. In other words, if there is no change in the parameter (i.e. it is essentially constant) or the change is within a medically acceptable tolerance, it may be concluded that the liver function is maintained.

Physiological parameters, even in healthy subjects, are often not entirely constant and there may be changes associated with a circadian rhythm, changes resulting from the type of food that was consumed, changes arising out of treatment by other medications; exposure to pathogens, etc. Thus, at times, changes in physiological parameters may occur but be within a certain acceptable range and be regarded as being substantially the same. In the context of the present disclosure, the term “medically acceptable tolerance” relates to measured changes that may be attributed to other causes and may thus be regarded from a physician's medical point of view as being substantially the same. The medically acceptable tolerance may, for example and depending on the parameter, be a change that is less than 50%, less than 40%, less than 30%, less than 20%, less than 10%, even less than 5% and, at times even less than 2% difference, 1% difference or no measurable difference in the parameter between the two or more time points. In this context, a parameter that is essentially constant is understood to encompass also changes in the level of the parameter that are medically tolerated.

In one embodiment, the physiological parameter is determined at least once after (immediately after, several hours after and even a day or more after) the subject having chronic liver disease is provided with an amount of Cl-IB-MECA. In some other embodiments, the physiological parameter is determined after two or more doses of Cl-IB-MECA are provided to the subject.

The chronic liver disease may involve a cirrhotic liver and/or a fibrotic liver. In one preferred embodiment, the chronic liver disease is cirrhosis.

Chronic liver disease or cirrhosis may be caused by or be associated with various medical conditions. Without being limited thereto, such conditions may include chronic alcohol consumption, chronic liver infection, chronic liver inflammation, inflammatory disorder, autoimmune disorder, drug-induced liver injury, nonalcoholic metabolic disorder, chronic liver injury, hepatocellular carcinoma, adverse hepatic reactions upon use of dietary supplements.

Liver infection may be of any type known in the art to cause injury to liver tissue. In some embodiments, the chronic liver is chronic hepatitis B, C or D. Chronic hepatitis B or C cause inflammation that over time damages the liver and leads to cirrhosis. Hepatitis D also causes cirrhosis, but will occur only in subjects who already have hepatitis B.

In yet some other embodiments, the chronic liver infection are any one of HIV infection, cytomegalovirus, Epstein-Barr virus.

The chronic liver disease may also be drug induced as a result of long term drug treatment. Drugs inducing liver damage may be grouped according to the type of enzyme manifesting marked level elevation (indicative of liver damage). For example, hepatocellular hepatotoxicity, which may be a result from drugs such as acetaminophen and isoniazid; Cholestatic hepatotoxicity which is characterized by development of pruritus and jaundice accompanied by marked elevation of serum alkaline phosphatase levels, may be a result from drugs such amoxicillin/clavulanic acid and chlorpromazine.

Preferred examples of drugs that may cause chronic liver disease, particularly, cirrhosis include Amiodarone, Methotrexate and Nitrofurantoin.

Yet, in some embodiments, the chronic liver disease is an inflammatory or autoimmune disorder. The inflammatory or autoimmune disorder may be selected, without being limited thereto, chronic pancreatitis, inflammatory bowel disease, primary sclerosing cholangitis, primary biliary cirrhosis, primary hepatothiasis and recurrent pyogenic cholangitis, systemic lupus erythematosus, Celiac disease, hypothyroidism, Raynaud's phenomenon, Sicca syndrome.

In yet some embodiments, the chronic liver disease is a metabolic disorder, such as, without being limited thereto, diabetes, obesity, steatosis and non-alcoholic steatohepatitis, haemochromatosis, cystic fibrosis, alpha-1 antitrypsin deficiency, galactosemia, glycogen storage diseases, Wilson's disease, and intrahepatic cholestasis. Some of these causes are also recognized as inherited diseases. As noted above, the present disclosure also concerns treatment of liver cancer, as part of the treatment aspect disclosed herein. Specifically, it has been found that there is an essentially linear dose response to escalating amounts of Cl-IB-MECA. Further, it has been found that doses of Cl-IB-MECA that were found effective against cancer, were much higher than the doses acceptable for treatment with the structurally related derivative, IB-MECA.

As such, in accordance with the treatment aspect, there is provided a method for treating hepatocellular carcinoma (HCC) comprising administering to the subject being diagnosed as having HCC, a daily treatment dose Cl-IB-MECA being higher than the amount acceptable for IB-MECA treatment, the amount being above 10 mg/day.

With respect to both aspects disclosed herein, the Cl-IB-MECA is used in an amount that are considered safe, tolerable and within dose limiting toxicity for oral administration to a subject, preferably human subject. Further, Cl-IB-MECA is provided at concentrations that show medically beneficiary pharmacokinetics, and with minimal side effects.

Specifically, as is demonstrated in the non-limiting examples provided herein, Cl-IB-MECA was found to be even safer than the alternative sorafenib or to other drugs given to patients with cirrhosis and/or hepatic impairment.

When referring to the treatment aspect, it was even striking to find that Cl-IB-MECA was effective in treatment of Child Pugh B patients, as such patients treated with Sorafenib generally have poor outcomes doe to underlying liver dysfunction. Thus, the results lead to the conclusion that Cl-IB-MECA is unexpectedly suitable for treatment of this sub-population.

In accordance with the treatment aspect, there is also provided a package comprising a pharmaceutical composition comprising as active ingredient Cl-IB-MECA and instructions for use of the Cl-IB-MECA for treatment of HCC, the instructions comprising administering to a subject in need of the treatment a daily treatment dose of Cl-IB-MECA effective to treat HCC. The instruction may also comprise treatment regimen of the daily treatment dose, e.g. the number of dosage unit forms to be given to the patient in order to receive the prescribed daily treatment dose. For example, the instructions may provide for a treatment regimen where the subject is to be given a daily treatment dose of 20 mg in two dosage unit forms, each comprising 10 mg.

The amount effective to maintain liver function or treat HCC may be readily determined, in accordance with the present disclosure, by administering to a plurality of tested subjects various amounts of the Cl-IB-MECA and then plotting the physiological response (for example an integrated “SS index” combining several physiological parameters indicative of liver function) as a function of the amount. Alternatively, the effective amount may also be determined, at times, through experiments performed in appropriate animal models and then extrapolating to human beings using one of a plurality of conversion methods; or by measuring the plasma concentration or the area under the curve (AUC) of the plasma concentration over time and calculating the effective dose so as to yield a comparable plasma concentration or AUC. As known, the effective amount may depend on a variety of factors such as mode of administration (for example, oral administration may require a higher dose to achieve a given plasma level or AUC than an intravenous administration); the age, weight, body surface area, gender, health condition and genetic factors of the subject; other administered drugs; etc.

In accordance with the maintenance aspect, the amount determined to be effective is between 1 μg/kg body weight to 1 mg/kg body weight. At times, the amount is between 10 μg/kg body weight and 500 μg/Kg body weight. In some embodiments, the amount is any one of 1, 2, 5, 10, 25, 50, 100, 150, 200, 500, 800 μg/Kg body weight and ranges therebetween. These amounts may be converted to total daily treatment doses by multiplication by body weight. Typically, the conversion is by multiplication by 70 kg (being the average of a body weight of an adult subject). As such, 1 μg/kg body weight is equivalent to a dose of 0.07 mg.

In accordance with the treatment aspect, the amount effective to treat HCC is at least 10 mg for daily administration. In some other embodiments, the amount, namely, the daily treatment dose is 15 mg, 20 mg, 25, 30 mg, 35 mg, 40 mg, 45 mg, 50 mg or any amount within the range of 10 mg and 100 mg.

In the context of the treatment aspect, the term “treatment” is to be understood as any effect resulting from administration of Cl-IB-MECA in the daily treatment dose on the tumor or symptoms associated with the tumor, these include, without being limited thereto, survival of the subjects being treated, partial or full shrinkage of the tumor, elimination of the tumor, amelioration of symptoms indicative of shrinkage or elimination of the tumor including the subjects general well being, and improvement in one or more physiological parameters indicative of liver dysfunction.

When referring to “survival”, it is to be understood as encompassing cure from the disease, namely, where diagnosis by conventional methods show that the tumor was eliminated; as well as prognosis-free survival rate, namely, the length of time during and after the treatment of HCC with Cl-IB-MECA that the patient lives with the disease but it does not get worse.

The Cl-IB-MECA may be provided to the subject as a single dose or in the course of long term treatment.

In some embodiments, Cl-IB-MECA is provided to the subject on a daily bases, once, twice or thrice per day. Thus, the above recited amounts are considered daily amounts.

The Cl-IB-MECA may be used together with a pharmaceutically acceptable carrier, namely an inert, non-toxic material, which does not react with Cl-IB-MECA and which can be added to formulations as diluents or carriers or to give form or consistency to the formulation. The carrier may include additives, colorants, diluents, buffering agents, disintegrating agents, moistening agents, preservatives, flavoring agents, and pharmacologically compatible carriers. In the case where the administration is oral, the carrier is one that is acceptable for oral administration, and in case of injection, the carrier is one that is acceptable for administration by injection. Injection may include, without being limited thereto,

An oral formulation, may be in the form of a pill, capsule, in the form of a syrup, an aromatic powder, and other various forms. A formulation for injection may be in a form suitable for intraperitoneal (i.p) injection, intravenous (i.v.) injection or local injection (directly into the liver).

The carrier may also, at times, have the effect of improving the delivery or penetration of the Cl-IB-MECA to the liver, for improving the stability of the Cl-IB-MECA, for slowing clearance rates of the Cl-IB-MECA, for imparting slow release properties of the Cl-IB-MECA, for reducing undesired side effects etc. The carrier may also be a substance that stabilizes the formulation (e.g. a preservative), for providing the formulation with an edible flavor, etc.

As used herein, the forms “a”, “an” and “the” include singular as well as plural references unless the context clearly dictates otherwise. For example, the term “a compound” includes one or more compounds.

Further, as used herein, the term “comprising” is intended to mean that the composition include the recited active agent, i.e. Cl-IB-MECA, but not excluding other elements, such as other active agents. The term “consisting essentially of” is used to define compositions which include the recited elements but exclude other elements that may have an essential significant effect on the performance (e.g. delivery) of Cl-IB-MECA. “Consisting of” shall thus mean excluding more than trace elements of other elements. Embodiments defined by each of these transition terms are within the scope of this invention.

Further, all numerical values, e.g. when referring the amounts or ranges of the elements constituting the composition comprising Cl-IB-MECA as an active ingredient, are approximations which are varied (+) or (−) by up to 20%, at times by up to 10% of from the stated values. It is to be understood, even if not always explicitly stated that all numerical designations are preceded by the term “about”.

The invention will now be exemplified in the following description of experiments that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### DESCRIPTION OF A NON-LIMITING EXAMPLE

### Example 1

### Long Term Maintenance of Liver Function in Patients Having Advanced Hepatocellular Carcinoma

### Study Group:

All patients were over the age of 18 with incurable advanced, refractory, or metastatic hepatocellular carcinoma (HCC), documented by cytology and/or histology for patients without underlying cirrhosis or according to the American Association for the Study of Liver Diseases Practice Guideline algorithm for patients with underlying cirrhosis.

All patients had measurable disease, according to Response Evaluation Criteria in Solid Tumors (RECIST); Eastern Collaborative Oncology Group (ECOG) performance status of 0, 1, or 2 at baseline; cirrhosis status Child-Pugh A or B; adequate renal and hematological function; negative pregnancy test for women of childbearing potential; and life expectancy of ≧12 weeks.

All patients provide written informed consent to participate in the study, which was approved by the investigational site's Independent Ethics Committee and the Israeli Ministry of Health.

Exclusion criteria included any chemotherapy, immunomodulatory drug therapy, immunosuppressive therapy, corticosteroids >20 mg/day prednisone or equivalent, or growth factor treatment (eg, erythropoietin) within 14 days prior to initiation of study drug; major surgery or radiation therapy within 28 days prior to initiation of study drug; severe liver dysfunction (Child-Pugh Class C or hepatic encephalopathy); active infection requiring systemic therapy; and uncontrolled congestive heart failure (New York Heart Association Classification 3 or 4), angina, myocardial infarction, cerebrovascular accident, coronary/peripheral artery bypass graft surgery, transient ischemic attack, or pulmonary embolism within 3 months prior to initiation of study drug

Demographic and baseline subject characteristics data appear in Table 1.

TABLE 1
Demographics and Baseline Characteristics:

| | | |
|---|---|---|
| Median age (years) | 71 | (60-89) |
| Male/Female | 11/7 | (61%/39%) |
| History of Hepatitis B | 8 | (44%) |
| History of Hepatitis C | 10 | (56%) |
| Child-Pugh A | 13 | (72%) |
| Child-Pugh B | 5 | (28%) |
| Prior surgery for HCC | 4 | (22%) |
| Prior radiotherapy | 3 | (17%) |
| Prior chemoembolization | 8 | (44%) |
| Prior targeted therapy (sorafenib) | 12 | (67%) |

### Objectives:

A Phase 1-2, open-label, dose-escalation study evaluating the safety, tolerability, pharmacokinetics, and pharmacodynamics of orally administered Cl-IB-MECA (CF102) in patients with advanced hepatocellular carcinoma (HCC) was conducted.

The primary objectives of the study were to determine the safety and tolerability, dose-limiting toxicities (DLTs), maximum tolerated dose (MTD), and recommended Phase 2 dose (RP2D) of orally administered CF102 in patients with advanced HCC, and to assess the repeat-dose PK behavior of CF102 in those patients.

The study was conducted in compliance with Good Clinical Practices, investigational site Institutional Review Board Regulations, Informed Consent Regulations, and the Declaration of Helsinki.

### Materials:

The A₃AR agonist, 2-Chloro-N⁶-(3-iodobenzyl)-adenosine-5′-N-methyluronamide (Cl-IB-MECA, referred to herein by the abbreviated name CF102), synthesized for Can-Fite BioPharma by Albany Molecular Research Inc, Albany, N.Y., USA.

### Methods:

The trial employed a conventional “three plus three” design. Subjects were treated with oral doses of CF102 in consecutive, 28-day cycles. The initial dose of CF102 was 1 mg twice daily (BID), with subsequent escalations to 5 mg and 25 mg BID, unless limited by toxicity. Subjects were evaluated weekly for the first cycle, every 2 weeks for cycles 2 and 3, and at the end of each subsequent cycle, up to 6 cycles of CF102 treatment. At the first stage nine patients were enrolled for the dose escalation phase and then additional nine were enrolled for the dose confirmation phase. Furthermore, nine patients have undergone intra-subject dose escalation per protocol. Subjects returned for a follow-up visit 28 days after completion of the last dose of study drug. Subjects who tolerate the drug and whose HCC did not progress were allowed to continue to receive CF102 at the discretion of the investigator and with approval from the Sponsor (e.g., in an extension protocol).

CF102 was escalated in successive cohorts of 3 subjects per dose level. If none of the 3 subjects at a dose level experience first cycle DLT, then 3 new subjects were entered at the next higher dose level.

The safety and the MTD of CF102 was determined by characterization of DLTs; characterization of the type, incidence, severity (graded by Common Terminology Criteria for Adverse Events (CTCAE, version 4.0), timing, seriousness, and relationship to treatment of adverse events (AEs); effects on vital signs and laboratory parameters; and changes in electrocardiograms (ECGs). The clinical activity of CF102 was determined by x-ray or appropriate imaging techniques (magnetic resonance imagining, unless computed tomography scan was appropriate) and RECIST evaluation every 2 cycles. Pharmacokinetic parameters included area under the curve (AUC), maximum plasma concentration (Cmax), through plasma concentration (Cmin), time to maximum plasma concentration (Tmax), and plasma half-life (t ½).

### Statistical Analysis

Safety data analysis was conducted on all subjects who received at least one dose of CF102. The number and percentage of subjects who experienced one or more AEs were summarized by dose level group, relationship to study drug, and severity. AEs were coded using Medical Dictionary for Regulatory Activities (MedDRA) terminology. Clinical laboratory parameters were summarized using descriptive statistics, by post-dosing shifts relative to baseline, and data listings of clinically significant abnormalities. Vital signs and ECG data were summarized by changes from baseline values at each dose level using descriptive statistics. Pharmacokinetic parameters, including AUC (0-8 h), Cmax, Cmin, and Tmax was estimated. Comparisons across dose levels were made to assess proportionality. Overall survival was calculated using the Kaplan-Meier method.

### Results:

### 1. Safety Data—CF102 is Safe and Well Tolerated

Overall excellent safety was recorded with no changes in vital signs, hematological or the chemistry parameters.

The moderate to severe adverse events experience to date vary. In the present study, the most commonly reported events were those related to loss of appetite; ascites or abdominal swelling; nausea or vomiting; diarrhea, constipation, and other gastrointestinal symptoms; edema; cellulitis; cerebral hemorrhage; headache, hyponatremia, leg thrombus; and musculoskeletal pain. Many of these events are expected in a population of patients with advanced HCC.

### 2. Pharmacokinetics—CF102 has Good Oral Bioavailability

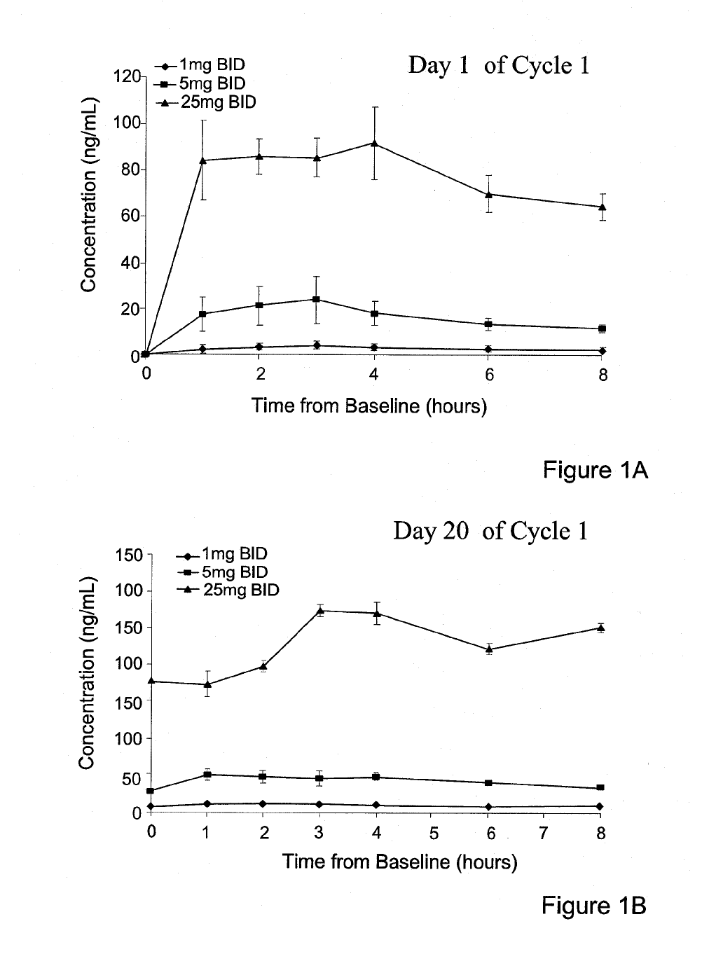

CF102 has shown good oral bioavailability and linear PK behavior given at single (FIG. 1A) or repeated (FIG. 1B) doses. Plasma levels at all doses are beyond the affinity value of CF102 at the A3AR (0.66 nM).

### 3. Efficacy, Overall Survival (OS) Data

The median OS of the 18 subjects to date has been 7.82 months (range, 3.5-27.3 months) with one subject receiving CF102 for 41+ months and continuing on drug. An additional important finding of this study is the 7 month median OS (range, 3.5-13.2 months) of 12 patients who were sorafenib failures and were treated with CF102 as second line therapy. Also, the median OS of the five Child-Pugh B patients was 8.1 months (range, 7.0-13.2 months) (Table 2).

TABLE 2
Overall survival data

| | | Months | Months | | Survival |
|---|---|---|---|---|---|
| | Starting | from | on | Entry | from start |
| Subject | Dose | diagnosis | prior | Child-Pugh | of CF102 |
| No. | (mg BID) | to CF102 | sorafenib | Class | (months) |
| 302 | 1 | 27 | — | A | 8.2 |
| 303 | 1 | 8 | — | A | 27.3* |
| 304 | 1 | 5 | — | A | 7.1 |
| 305 | 5 | 18 | 8.2 | A | 4.1 |
| 306 | 5 | 14 | 7.0 | A | 7.1 |
| 307 | 5 | 24 | 1.4 | A | 10.6 |
| 308 | 25 | 14 | 10.8 | A | 8.3 |
| 309 | 25 | 1 | — | B | 8.1 |
| 310 | 25 | 1 | — | B | 7.6 |
| 312 | 5 | 20 | — | B | 10.7 |
| 313 | 25 | 14 | 3.3 | B | 7.0 |
| 314 | 1 | 53 | 4.8 | A | 5.6 |
| 315 | 5 | 22 | 8.0 | A | 3.5 |
| 316 | 25 | 78 | 2.3 | A | 12.6 |
| 317 | 1 | 26 | 5.0 | B | 13.2** |
| 318 | 1 | 33 | 24.2 | A | 7.0 |
| 319 | 5 | 19 | 2.6 | A | 4.2 |
| 320 | 25 | 19 | 6.0 | A | 8.4 |

| Median | All | 7.8 |
|---|---|---|
| | Sorafenib failures | 7.0 |
| | Child Pugh B | 8.1 |

Analysis of CF102 treatment duration by dose shows that the longest time period on CF102 treatment was in the 25 mg dose (61.2 months). Patients were treated with the 5 mg and 1 mg dose for 49.9 and 24.2 months, respectively.

These data are particularly striking when taken in the context of prior experience with comparable patient populations as presented by WÖrns et al. [WÖrns et al. Oncology 2010; 79:85-92] showing, in Table 2 of its publication that the median OS of patients with Child-pugh B treated with Sunitinib was 3.5 months in comparison to 8.1 months in the present CF102 study. Taking into consideration that Child-pugh B patients are not eligible to be treated with Sorafenib due to their liver condition, CF102 may be a preferable drug for this patient sub-population.

Additionally, one subject in the present CF102 trial experienced a 10 months period of Stable Disease (SD) by RECIST criteria.

### 4. CF102 Maintained Liver Function

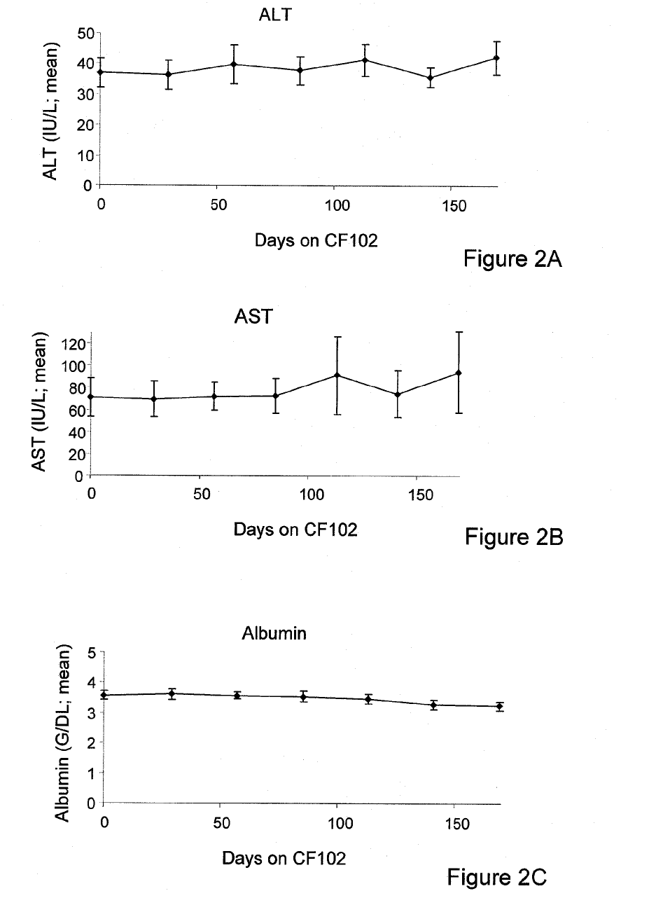

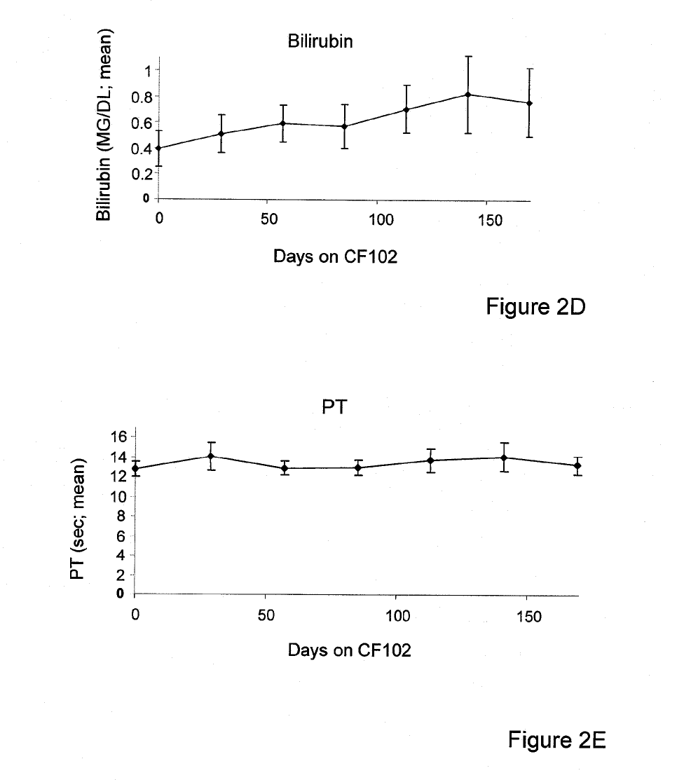

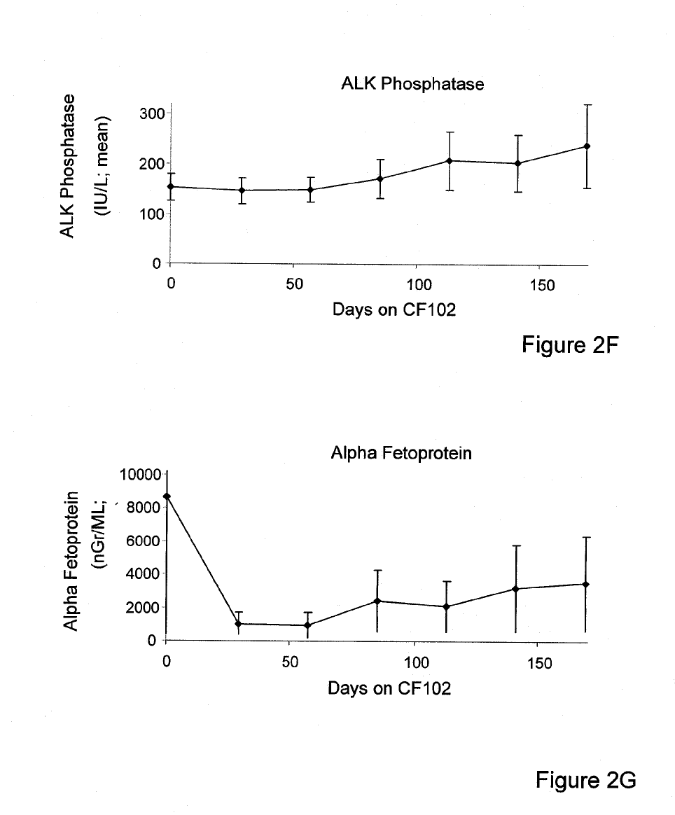

The therapeutic effect of CF102 was also demonstrated by the analysis of a physiological parameter over a period of 6 months, the parameters, including serum level of liver enzymes, such as alanine transaminase (ALT), aspartate transaminase (AST), albumin, bilirubin, prothrombin time (PT), ALK phosphatase (ALP) and α-fetoprotein over a 6 months period. The data revealed that the different parameters remained stable, suggesting that CF102 was responsible for maintaining the liver function (FIG. 2A-2G).

The data provided in FIGS. 2A to 2G shows that the different parameters remained stable, suggesting that CF102 is effective for maintaining liver function in chronic liver disease and cirrhotic liver.

1-47. (canceled)

48. A method for long term maintenance of liver function in a subject having a chronic liver disease, the method comprising administering to the subject having said chronic liver disease an effective amount of 2-Chloro-N⁶-(3-iodobenzyl)-adenosine-5′-N-methyluronamide (Cl-IB-MECA).

49. The method of claim 48, wherein said amount is effective to maintain, between at least two time points, at least one of which is after administration of said Cl-IB-MECA, an essentially constant level, within a medically acceptable tolerance, of at least one physiological parameter indicative of liver function.

50. The method of claim 48, wherein the chronic liver disease is cirrhosis.

51. The method of claim 48, wherein the liver disease is associated with or caused by one or more of condition selected from the group consisting of chronic alcohol consumption, long-term liver infection, chronic liver inflammation, inflammatory disorder, autoimmune disorder, drug-induced liver injury, nonalcoholic metabolic disorder, chronic liver injury, hepatocellular carcinoma, adverse hepatic reactions upon use of dietary supplements.

52. The method of claim 49, wherein the physiological parameter is a serum level of one or more of a liver enzyme, albumin and glucose.

53. The method of claim 49, wherein the physiological parameter is a serum level of one or more of alanine transaminase (ALT) and aspartate transaminase (AST), albumin, bilirubin, prothrombin time (PT), ALK phosphatase and α-fetoprotein.

54. The method of claim 49, wherein at least one of the time points is after administration to the subject Cl IB MECA.

55. The method of claim 49, comprising oral administration of said Cl-IB-MECA.

56. The method of claim 55, comprising administration of Cl-IB-MECA once or twice a day.

57. The method of claim 56, comprising administration of Cl-IB-MECA twice a day.

58. The method of claim 48, comprising administration of Cl-IB-MECA in a daily amount of between 1 μg/kg body weight and 1 mg/Kg body weight.

59. The method of claim 58, wherein the daily amount is between 10 μg/kg body weight and 500 μg/Kg body weight.

60. The method of claim 49, wherein the liver disease is associated with or caused by one or more of condition selected from the group consisting of chronic alcohol consumption, long-term liver infection, chronic liver inflammation, inflammatory disorder, autoimmune disorder, drug-induced liver injury, nonalcoholic metabolic disorder, chronic liver injury, hepatocellular carcinoma, adverse hepatic reactions upon use of dietary supplements.

61. The method of claim 49, comprising administration of Cl-IB-MECA in a daily amount of between 1 μg/kg body weight and 1 mg/Kg body weight.

62. The method of claim 61, wherein the daily amount is between 10 μg/kg body weight and 500 μg/Kg body weight.
